(19) United States
(12) Patent Application Publication
(10) Pub. No.: US 2026/0191760 A1
(43) Pub. Date: Jul. 9, 2026

(54) COSMETIC COMPOSITION

(71) Applicant: Shiseido Company, Ltd., Chuo-ku, Tokyo (JP)

(72) Inventors: Takashi OKA, Tokyo (JP); Hiroko MANABE, Tokyo (JP); Saori IETANI, Tokyo (JP); Yuji ITO, Tokyo (JP)

(73) Assignee: Shiseido Company, Ltd., Chuo-ku, Tokyo (JP)

(21) Appl. No.: 19/133,394

(22) PCT Filed: Dec. 8, 2023
(86) PCT No.: PCT/JP2023/043990

§ 371 Date: May 28, 2025

(30) Foreign Application Priority Data
Dec. 28, 2022 (JP) 2022-212581

(51) Int. Cl. A61K 8/86; A61K 8/368; A61K 8/44; A61K 8/49; A61K 8/67; A61Q 19/00

(52) U.S. Cl. CPC A61K 8/86; A61K 8/368; A61K 8/44; A61K 8/4926; A61K 8/673; A61Q 19/00

(57) ABSTRACT

Provided are a novel percutaneous penetration enhancer for a water-soluble active component and a cosmetic composition comprising the novel percutaneous penetration enhancer.

A composition that is a cosmetic composition comprising a polymer having a structure represented by formula (1) below and a water-soluble active component,

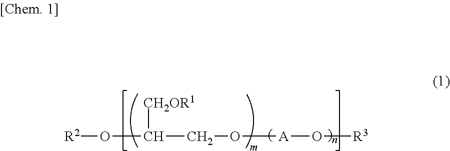

where in the formula (1), R¹, R², and R³ are each independently a hydrogen atom or an alkyl group having 1 to 4 carbon atoms; A is an alkylene group having 2 to 4 carbon atoms; and m and n are each independently 1.0 to 50, and wherein
a number average molecular weight of the polymer is 10,000 or less, and
an IOB value of the polymer is 0.4 to 1.8.

### FIELD

The present invention relates to a cosmetic composition.

### BACKGROUND

The stratum corneum, as the outermost layer of the skin, has a hydrophobic barrier function, and thus has a property of inhibiting penetration of moisture and water-soluble active components.

Until now, a large number of percutaneous penetration enhancers or cosmetic compositions comprising such percutaneous penetration enhancers have been developed for allowing moisture and water-soluble active components to effectively penetrate into the skin.

For example, PTL 1 discloses an external preparation for skin, comprising an alkylene oxide derivative represented by general formula (I) below, and discloses that the alkylene oxide derivative has an effect of enhancing percutaneous absorption:

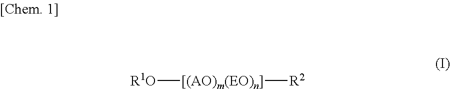

PTL 2 discloses a beauty composition suitable for topical application, as a beauty composition that enhances penetration of active components into the skin and extends residency time of active components within the skin, comprising i) glycerin; ii) a lipid bilayer structurant with a glyceryl headgroup; iii) a penetration enhancer; and iv) a skin care active component.

PTL 3 discloses a skin penetration enhancer for (a) at least one hydroxy acid, for the purpose of increasing skin penetration of a hydroxy acid, comprising (b) at least one basic amino acid; (c) at least one amino acid surfactant selected from the group consisting of N-acyl aspartate and derivatives thereof; and (d) water.

### CITATION LIST

### Patent Literature

[PTL 1] Japanese Unexamined Patent Publication (Kokai) No. 2004-083541
[PTL 2] Japanese Unexamined PCT Publication (Kohyo) No. 2017-500322
[PTL 3] Japanese Unexamined Patent Publication (Kokai) No. 2019-116421

### SUMMARY

### Technical Problem

Although the effects of specific components on penetration into the skin have been studied to some extent, the penetration of water-soluble active components into the skin remains to have room for improvement.

The present invention aims to improve the above circumstances, and an object thereof is to provide a novel percutaneous penetration enhancer for a water-soluble active component and a cosmetic composition comprising the novel percutaneous penetration enhancer.

### Solution to Problem

The present invention that achieves the above object is as follows.

### <Aspect 1>

A composition that is a cosmetic composition comprising a polymer having a structure represented by formula (1) below and a water-soluble active component,

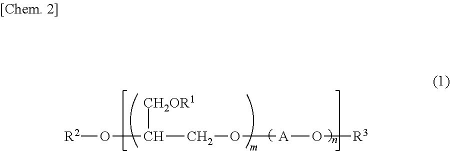

where in the formula (1), R¹, R², and R³ are each independently a hydrogen atom or an alkyl group having 1 to 4 carbon atoms; A is an alkylene group having 2 to 4 carbon atoms; and m and n are each independently 1.0 to 50,
wherein a number average molecular weight of the polymer is 10,000 or less, and
wherein an IOB value of the polymer is 0.4 to 1.8.

### <Aspect 2>

The composition according to Aspect 1, wherein
in the formula (1), A is represented by formula (2) below:

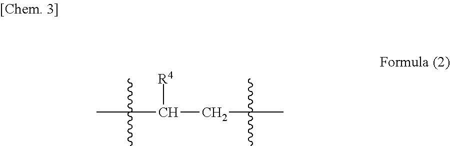

where in the formula (2), R⁴ is an alkyl group having 1 or 2 carbon atoms.

### <Aspect 3>

The composition according to Aspect 1 or 2, wherein the polymer is a random copolymer.

### <Aspect 4>

The composition according to any one of Aspects 1 to 3, wherein in the formula (1), m is 2 or greater and at least a portion of R includes a methyl group.

### <Aspect 5>

The composition according to any one of Aspects 1 to 4, wherein in the formula (1), at least one of R² and R³ is a methyl group.

### <Aspect 6>

The composition according to any one of Aspects 1 to 5, wherein the water-soluble active component comprises an ionic substance, or a nonionic substance having a log P value of less than 2.0.

### <Aspect 7>

The composition according to any one of Aspects 1 to 6, wherein the water-soluble active component comprises at least one selected from a group consisting of alkoxysalicylic acid or salts thereof, 1-piperidinepropionic acid or salts thereof, B vitamins or derivatives thereof, and amino acids or derivatives thereof.

### <Aspect 8>

The composition according to any one of Aspects 1 to 7, wherein the polymer is a percutaneous penetration enhancer of the water-soluble active component.

### <Aspect 9>

Use of a polymer having a structure represented by formula (1) below, as a percutaneous penetration enhancer of a water-soluble active component:

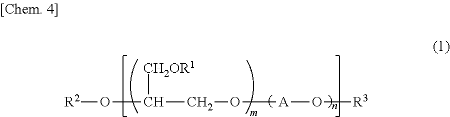

where in the formula (1), R¹, R², and R³ are each independently a hydrogen atom or an alkyl group having 1 to 4 carbon atoms; A is an alkylene group having 2 to 4 carbon atoms; and m and n are each independently 1.0 to 50,
wherein a number average molecular weight of the polymer is 10,000 or less, and
wherein an IOB value of the polymer is 0.4 to 1.8.

### <Aspect 10>

The use according to Aspect 9, wherein
in the formula (1), A is represented by formula (2) below:

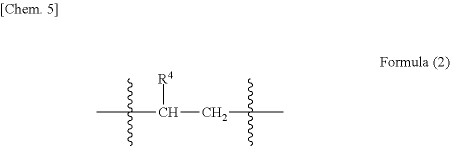

where in the formula (2), R⁴ is an alkyl group having 1 or 2 carbon atoms.

### <Aspect 11>

The use according to Aspect 9 or 10, wherein the polymer is a random copolymer.

### <Aspect 12>

The use according to any one of Aspects 9 to 11, wherein in the formula (1), m is 2 or greater and at least a portion of R¹ includes a methyl group.

### <Aspect 13>

The use according to any one of Aspects 9 to 12, wherein in the formula (1), at least one of R² and R³ is a methyl group.

### <Aspect 14>

The use according to any one of Aspects 9 to 13, wherein the water-soluble active component comprises an ionic substance, or a nonionic substance having a log P value of less than 2.0.

### <Aspect 15>

The use according to any one of Aspects 9 to 14, wherein the water-soluble active component comprises at least one selected from a group consisting of alkoxysalicylic acid or salts thereof, 1-piperidinepropionic acid or salts thereof, B vitamins or derivatives thereof, and amino acids or derivatives thereof.

### Advantageous Effects of Invention

According to the present invention, a novel percutaneous penetration enhancer for a water-soluble active component and a cosmetic composition comprising the novel percutaneous penetration enhancer can be provided.

### BRIEF DESCRIPTION OF DRAWINGS

FIG. 1 is a chart showing the results of cumulative permeation amounts of the water-soluble active component of Example 1, Example 2, and Comparative Example 1.

FIG. 2 is a chart showing the results of cumulative permeation amounts of the water-soluble active component of Example 3 and Comparative Example 2.

FIG. 3 is a chart showing the results of cumulative permeation amounts of the water-soluble active component of Example 4, Example 5, and Comparative Example 3.

FIG. 4 is a chart showing the results of cumulative permeation amounts of D-glutamic acid contained in the water-soluble active component of Example 6, Example 7, and Comparative Example 4.

FIG. 5 is a chart showing the results of cumulative permeation amounts of DL-alanine contained in the water-soluble active component of Example 6, Example 7, and Comparative Example 4.

FIG. 6 is a chart showing the results of cumulative permeation amounts of DL-methionine contained in the water-soluble active component of Example 6, Example 7, and Comparative Example 4.

### DESCRIPTION OF EMBODIMENTS

Hereinafter, embodiments of the present invention will be described in detail. Note that the present invention is not limited to the following embodiments, and various modifications can be made within the scope of the invention.

### <<Cosmetic Composition>>

The cosmetic composition of the present invention (hereinafter, also simply referred to as “composition of the present invention”) is a composition that is
a cosmetic composition comprising a polymer having a structure represented by formula (1) below (hereinafter, also simply referred to as “polymer of the present invention”) and a water-soluble active component,

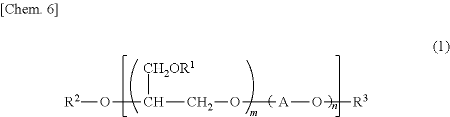

where in the formula (1), R¹, R², and R³ are each independently a hydrogen atom and an alkyl group having 1 to 4 carbon atoms; A is an alkylene group having 2 to 4 carbon atoms; and m and n are each independently 1.0 to 50,
wherein a number average molecular weight of the polymer is 10,000 or less, and
wherein an IOB value of the polymer is 0.4 to 1.8.

### <Polymer of Present Invention>

The composition of the present invention comprises the polymer of the present invention described below. The polymer of the present invention has an effect of improving penetration of a water-soluble active component into the skin. Therefore, it can be said that the polymer of the present invention is a percutaneous penetration enhancer of a water-soluble active component. In other words, the polymer of the present invention can be used as a percutaneous penetration enhancer of a water-soluble active component contained in the composition of the present invention.

Without wishing to be bound by theory, it is presumed that this is derived from the above characteristic of the polymer of the present invention: the unique structure indicated by the formula (1), number average molecular weight in a specific range (10,000 or less), and IOB (inorganic organic balance) value in a specific range (0.4 to 1.8).

Based on intensive studies by the present inventors, it was found that the polymer of the present invention can interact with stratum corneum intercellular lipids. Stratum corneum intercellular lipids generally hinder penetration of substances such as water-soluble active components into the skin. However, it is presumed that the intercellular lipids, when interacting with the polymer of the present invention, enters a temporarily unstable state, and at this point, a water-soluble active component can more easily penetrate into the skin, i.e., penetration of the water-soluble active component is improved.

In the present invention, “enhancing percutaneous penetration” means enabling rapid penetration into the skin. Thus, when the “percutaneous penetration enhancer” of the present invention is used, compared to when not used, a water-soluble active component can penetrate more rapidly.

The polymer of the present invention has a structure represented by formula (1) below:

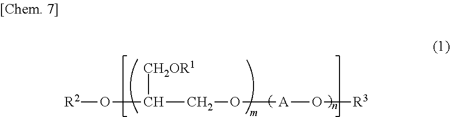

In the formula (1), R¹, R², and R³ are each independently a hydrogen atom or an alkyl group having 1 to 4 carbon atoms. In the present invention, examples of the alkyl group having 1 to 4 carbon atoms include a methyl group, an ethyl group, an n-propyl group, an isopropyl group, an n-butyl group, a sec-butyl group, and a tert-butyl group. These alkyl groups may further comprise a substituent as long as the effect of the present invention is not impaired. The substituent is not particularly limited, and examples include halogeno groups.

According to one embodiment of the present invention, in the formula (1), R¹, R², and R³ may each independently be a hydrogen atom, a methyl group, an ethyl group, an n-propyl group, an isopropyl group, an n-butyl group, a sec-butyl group, or a tert-butyl group.

According to one embodiment of the present invention, in the formula (1), R¹ is preferably a hydrogen atom, a methyl group, or an ethyl group. When m is 2 or greater, i.e., when a plurality of R¹ are present, each R¹ may be the same or different, and in particular, at least a portion of R¹ preferably includes a methyl group. The case where at least a portion of R¹ includes a methyl group is preferable, for example, from the viewpoint of improving feeling of use of the composition of the present invention.

According to one embodiment of the present invention, in the formula (1), R¹ may be in a state where a hydrogen atom and an alkyl group having 1 to 4 carbon atoms coexist. Specifically, one portion of R¹ may include a hydrogen atom, and the other portion thereof may include an alkyl group having 1 to 4 carbon atoms. Preferably, one portion of R¹ includes a hydrogen atom, and the other portion thereof includes a methyl group.

According to one embodiment of the present invention, in the formula (1), R² is preferably a hydrogen atom, a methyl group, or an ethyl group, and particularly, is more preferably a methyl group.

According to one embodiment of the present invention, in the formula (1), R² may be in a state where a hydrogen atom and an alkyl group having 1 to 4 carbon atoms coexist. Specifically, one portion of R² may include a hydrogen atom, and the other portion thereof may include an alkyl group having 1 to 4 carbon atoms. Preferably, one portion of R² includes a hydrogen atom, and the other portion thereof includes a methyl group.

According to one embodiment of the present invention, in the formula (1), R³ is preferably a hydrogen atom, a methyl group, or an ethyl group, and particularly, is more preferably a methyl group.

According to one embodiment of the present invention, in the formula (1), at least one of R² and R³ is preferably a methyl group.

In the formula (1), A is an alkylene group having 2 to 4 carbon atoms. More specifically, A may be, for example, an ethylene group, a propylene group, a butylene group, or an isobutylene group, but is not limited thereto. In particular, A is preferably represented by formula (2) below:

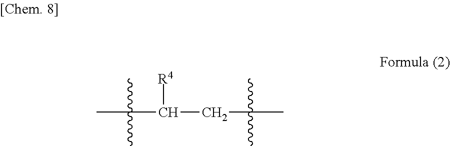

In the formula (2), R⁴ is an alkyl group having 1 or 2 carbon atoms. More specifically, R⁴ may be a methyl group or an ethyl group.

In the formula (1), m and n represent an average number of moles added for each structural unit. The m and n are each independently 1.0 to 50, and more specifically, may be 1.0 or greater, 1.5 or greater, 2.0 or greater, 3.0 or greater, 4.0 or greater, 5.0 or greater, 6.0 or greater, 7.0 or greater, 8.0 or greater, 9.0 or greater, or 10 or greater, and may be 50 or less, 45 or less, 40 or less, 35 or less, 34 or less, 32 or less, 30 or less, 28 or less, 26 or less, 25 or less, 24 or less, 22 or less, 20 or less, 18 or less, 16 or less, 15 or less, 14 or less, 12 or less, 10 or less, or 5.0 or less.

In the formula (1), m is preferably 1.0 or greater and 14 or less, and more preferably 2.0 or greater and 5.0 or less. In addition, in the formula (1), n is preferably 2.0 or greater and 34 or less, and more preferably 6.0 or greater and 12 or less.

According to one embodiment of the present invention, in the formula (1), m+n may be 4.0 or greater, 4.5 or greater, 5.0 or greater, 6.0 or greater, 7.0 or greater, 8.0 or greater, 9.0 or greater, 10 or greater, 11 or greater, 12 or greater, 13 or greater, 14 or greater, or 15 or greater, and may be 50 or less, 45 or less, 40 or less, 35 or less, 30 or less, 25 or less, 20 or less, 15 or less, or 12 or less.

According to one embodiment of the present invention, in the formula (1), min may be 1:10 to 10:1.

For the polymer of the present invention, the number average molecular weight is 10,000 or less. More specifically, the number average molecular weight of the polymer of the present invention, for example, may be 10,000 or less, 5,000 or less, 3,500 or less, 3,200 or less, 3,000 or less, 2,800 or less, 2,500 or less, 2,200 or less, 2,000 or less, 1,800 or less, 1,500 or less, 1,400 or less, 1,300 or less, 1,200 or less, 1,100 or less, 1,000 or less, 900 or less, 800 or less, 700 or less, or 600 or less, and may be 130 or greater, 150 or greater, 200 or greater, 250 or greater, 300 or greater, 350 or greater, 400 or greater, 450 or greater, 500 or greater, 550 or greater, 600 or greater, 650 or greater, 700 or greater, 750 or greater, 800 or greater, 850 or greater, 900 or greater, 950 or greater, or 1,000 or greater. In addition, the number average molecular weight of the polymer of the present invention is preferably 300 or greater and 3,500 or less, and more preferably 500 or greater and 1,200 or less.

The IOB value of the polymer of the present invention is 0.4 to 1.8. More specifically, the IOB value of the polymer of the present invention, for example, may be 0.4 or greater, 0.5 or greater, 0.6 or greater, 0.7 or greater, 0.8 or greater, 0.9 or greater, or 1.0 or greater, and may be 1.8 or less, 1.6 or less, 1.4 or less, or 1.2 or less. In addition, the IOB value of the polymer of the present invention is preferably 0.7 or greater and 1.2 or less.

The IOB value is an abbreviation for inorganic/organic balance, a value that represents a ratio of inorganic value to organic value, and acts as an indicator showing the degree of polarity of an organic compound. The IOB value, specifically, is represented as IOB value=inorganic value/organic value. Regarding each of the “inorganic value” and “organic value”, “inorganic value” and “organic value” are set according to various atoms or functional groups, such that, for example, the “organic value” for one carbon atom in a molecule is 20, and the “inorganic value” for one hydroxyl group is 100. The IOB value of an organic compound can be calculated by summing the “inorganic values” and “organic values” of all atoms and functional groups in the organic compound (refer to, for example, Yoshio Koda, “Organic Conceptual Diagrams—Fundamentals and Applications—”, pp. 11 to 17, Sankyo Publishing Co., Ltd., 1984). Note that regarding the method of determining IOB values, the method described in the Examples can be referenced.

The polymer of the present invention may be a block copolymer, or may be a random copolymer, but is preferably a random copolymer.

The manufacturing method for the polymer of the present invention is not particularly limited and, for example, may be carried out by addition polymerization of epoxides corresponding to each structural unit. The addition polymerization may be block polymerization, or may be random polymerization, but is preferably random polymerization.

The manufacturing method for the polymer of the present invention may further comprise further reacting an alkyl halide with the polymer obtained by the above addition polymerization. A hydroxyl group in a molecule of the polymer obtained via the reaction with an alkyl halide is blocked by the alkyl group, and as a result, hydrophobicity of the entire polymer can be adjusted as desired. In the present invention, blocking rate of hydroxyl group by alkyl group within a molecule of the polymer, for example, may be 30% or greater, 35% or greater, 40% or greater, 45% or greater, 50% or greater, or 55% or greater, and may be 60% or less, 59% or less, 58% or less, 57% or less, 56% or less, or 55% or less. Note that the blocking rate of hydroxyl group by alkyl group can be determined by the method described in the Examples.

The polymer of the present invention, as one of the characteristics thereof, has both high water solubility and high lipid solubility.

The degree of solubility in water of the polymer of the present invention can be, for example, 10% by mass or greater, 15% by mass or greater, 20% by mass or greater, 25% by mass or greater, 30% by mass or greater, 35% by mass or greater, 40% by mass or greater, 45% by mass or greater, or 50% by mass or greater.

As an indicator of lipid solubility of the polymer of the present invention, for example, degree of solubility in olive oil can be used. The degree of solubility in olive oil of the polymer of the present invention, for example, can be 0.01% by mass or greater, 0.05% by mass or greater, 0.10% by mass or greater, 0.50% by mass or greater, or 1.00% by mass or greater. Note that the upper limit value of the degree of solubility in olive oil of the polymer of the present invention is not particularly limited and, for example, may be 10% by mass or less.

In the composition of the present invention, the content of the polymer of the present invention is not particularly limited and, for example, may be 0.001% by mass or greater, 0.005% by mass or greater, 0.01% by mass or greater, 0.05% by mass or greater, 0.1% by mass or greater, 0.5% by mass or greater, 1.0% by mass or greater, 2.0% by mass or greater, 3.0% by mass or greater, 4.0% by mass or greater, or 5.0% by mass or greater, and may be 90% by mass or less, 80% by mass or less, 70% by mass or less, 60% by mass or less, 50% by mass or less, 40% by mass or less, 30% by mass or less, 20% by mass or less, 10% by mass or less, 8.0% by mass or less, 5.0% by mass or less, 3.0% by mass or less, or 1.0% by mass or less.

The polymer of the present invention, when used as a percutaneous penetration enhancer of a water-soluble active component, for example, may be 1.0 parts by mass or more or 5.0 parts by mass or more, and preferably 10 parts by mass or more, 50 parts by mass or more, 100 parts by mass or more, 130 parts by mass or more, 150 parts by mass or more, 200 parts by mass or more, 250 parts by mass or more, 300 parts by mass or more, 350 parts by mass or more, 400 parts by mass or more, 450 parts by mass or more, or 500 parts by mass or more, and may be 10000 parts by mass or less, 5000 parts by mass or less, or 3000 parts by mass or less, and preferably 1000 parts by mass or less, relative to a total of 100 parts by mass of the water-soluble active component.

### <Water-Soluble Active Component>

The composition of the present invention comprises a water-soluble active component. In the present invention, “water-soluble active component” refers to a component that is water-soluble and demonstrates a function thereof by penetrating into the skin. The function of the water-soluble active component means, but is not particularly limited to, an ability to bring about some effect, particularly an advantageous effect, to the skin (for example, a moisturizing effect, a firming effect, a whitening effect, an anti-wrinkle effect, or an anti-spot effect).

In the present invention, the water-soluble active component is not particularly limited and, for example, may include an ionic substance, or a nonionic substance having a log P value of less than 2.0.

An “ionic substance” includes salts of metal ions that completely ionize in water (for example, potassium 4-methoxysalicylate). In addition, a “metal ion” includes alkali metal ions and alkaline earth metal ions, and more specifically includes, but is not limited to, for example, sodium ion, potassium ion, and calcium ion.

A “log P value” represents a ratio of equilibrium concentrations of a substance dissolved in the two phases, octanol and water, and is an indicator showing the hydrophilicity or hydrophobicity of the component (nonionic substance). In particular, it is known that when the log P value is near 2.0, skin penetration of the component (nonionic substance) is maximized, and when the log P value is less than that, skin penetration of the component (nonionic substance) decreases (for example, “Non-formulation Parameters That Affect Penetrant-Skin-Vehicle Interactions and Percutaneous Absorption”, JE Grice et al., “Percutaneous Penetration Enhancers Drug Penetration Into/Through the Skin” (2017), pp. 45 to 75). Thus, in the present invention, the log P value of a nonionic substance as a water-soluble active component may be less than 2.0, 1.9 or less, 1.8 or less, 1.7 or less, 1.6 or less, 1.5 or less, 1.4 or less, 1.3 or less, 1.2 or less, 1.1 or less, 1.0 or less, 0.9 or less, 0.8 or less, 0.7 or less, 0.6 or less, 0.5 or less, 0.4 or less, 0.3 or less, 0.2 or less, 0.1 or less, 0.0 or less, −0.1 or less, −0.2 or less, −0.3 or less, −0.4 or less, −0.5 or less, −0.6 or less, −0.7 or less, −0.8 or less, −0.9 or less, −1.0 or less, −1.1 or less, −1.2 or less, −1.3 or less, −1.4 or less, −1.5 or less, −1.6 or less, −1.7 or less, −1.8 or less, −1.9 or less, or −2.0 or less. The lower limit value of the log P value is not particularly limited and, for example, may be −8.0 or greater, −7.0 or greater, −5.0 or greater, or −3.0 or greater.

The log P value can be obtained by referencing a database in which log P values of many compounds are listed, available from, for example, Daylight Chemical Information Systems, Inc. (Daylight CIS) or PubChem (National Center for Biotechnology Information). If no actual log P value is available, the value can be calculated with the program “CLOGP” (Daylight CIS).

In the present invention, specific examples of ionic substances used as water-soluble active components are shown below, but are not limited thereto. Specifically, examples of ionic substances include salts of alkoxysalicylic acid, in particular, metal ion salts of alkoxysalicylic acid (for example, potassium 4-methoxysalicylate), tranexamic acid salts, L-ascorbic acid salts, magnesium ascorbyl phosphate, dipotassium glycyrrhizinate, and ionic B vitamins such as vitamin B1 and vitamin B12.

In the present invention, specific examples of nonionic substances having a log P value of less than 2.0, used as water-soluble active components, are shown below, but are not limited thereto. Specifically, the nonionic substances having a log P value of less than 2.0, for example, may comprise at least one selected from the group consisting of alkoxysalicylic acid having a log P value of less than 2.0, B vitamins or derivatives thereof having a log P value of less than 2.0, tranexamic acid (log P value: −2.0), L-ascorbic acid (log P value: −1.6), arbutin (log P value: −0.7), L-ascorbic acid glucoside (log P value: −3.1), glycylglycine (log P value: −2.3), 1-(2-hydroxyethyl)-2-imidazolidinone (log P-1.4), value: adenosine (log P value: −1.1), DIPY (dimethylpyrazolyldimethylpyrimidine hydrochloride; log P value: 0.8), taurine (log P value: 1.4), trimethylglycine (log P value: 0.1), pyridoxine (log P value: −0.8), sodium pyrrolidone carboxylate (log P value: −0.8), 1PP (1-piperidinepropionic acid; log P value: −1.5), vitamin C (log P value: −2.2), vitamin C glucoside (log P value: −3.1), ethyl vitamin C (log P value: −1.5); moisturizers such as glycerin (log P value: −1.8), 1,3-butyleneglycol (log P value: −0.4), dipropylene glycol (log P value: −0.5), trehalose (log P value: −1.7), xylitol (log P value: −2.5), sodium lactate (log P value: −0.7), and urea (log P value: −2.1); hyaluronic acid (log P value: −7.4), and amino acids (log P values: −1.0 to −4.0) such as glutamic acid (log P value: −3.7), alanine (log P value: −3.0), and methionine (log P value: −1.9) and derivatives thereof (including optical isomers).

Among the above, in the present invention, the water-soluble active component preferably comprises at least one selected from the group consisting of alkoxysalicylic acid or salts thereof, 1-piperidinepropionic acid or salts thereof, B vitamins or derivatives thereof, and amino acids or derivatives thereof.

### (Alkoxysalicylic Acid and Salts Thereof)

The alkoxysalicylic acid used in the present invention is one in which a hydrogen atom at any of positions 3, 4, and 5 of salicylic acid is substituted with an alkoxy group. The alkoxy group as the substituent is preferably any of a methoxy group, an ethoxy group, a propoxy group, an isopropoxy group, a butoxy group, and an isobutoxy group, and is more preferably a methoxy group or an ethoxy group.

If specific compounds are exemplified as alkoxysalicylic acid, 3-methoxysalicylic acid (log P value: 1.9) is included therein.

The salts (ionic substances) of alkoxysalicylic acid are not particularly limited and examples include alkali metal salts and alkaline earth metal salts such as sodium salts, potassium salts, and calcium salts; and salts such as ammonium salts and amino acid salts.

### (1-Piperidinepropionic Acid and Salts Thereof)

1-Piperidinepropionic acid is a compound indicated by the following chemical formula:

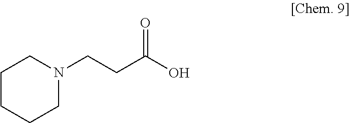 [SMILES: O=C(O)CCN1CCCCC1]

The salts (ionic substances) of 1-piperidinepropionic acid are not particularly limited and examples include alkali metal salts and alkaline earth metal salts such as sodium salts, potassium salts, and calcium salts; and salts such as ammonium salts and amino acid salts.

### (B Vitamins and Derivatives Thereof)

In the present invention, the B vitamins or derivatives thereof, for example, comprise one or more compounds selected from the group consisting of vitamin B1 (ionic substance), vitamin B2 (log P value: −1.5), vitamin B3 (also referred to as “niacin”), vitamin B5 (log P value: −1.1), vitamin B6 (log P value: −0.8), or vitamin B12 (ionic substance); or derivatives thereof, and in particular, preferably comprise vitamin B3, also called “niacin”, or a derivative thereof. Note that “niacin” includes nicotinic acid and niacinamide (also referred to as “nicotinamide”). From the viewpoint that niacinamide has both a whitening effect and an anti-wrinkle effect, in the present invention, it is preferable that the B vitamins or derivatives thereof be niacinamide (log P value: −0.4).

### (Amino Acids and Derivatives Thereof (Including Optical Isomers))

In the present invention, the amino acids and derivatives thereof (including optical isomers) are not particularly limited. More specifically, the amino acid, for example, may comprise at least one selected from glutamic acid (log P value: −3.7), alanine (log P value: −3.0), and methionine (log P value: −1.9).

A derivative of an amino acid refers to a substance having a slightly different structure but having the same or similar characteristics as the amino acid and, for example, may be an optical isomer (D-, L-, or DL-form) of the amino acid, or may be an amino acid in which an amino group or carboxy group is modified with a protecting group. However, among derivatives of amino acids, those that are ionic substances may be classified as the “ionic substance” described above.

In the composition of the present invention, the content of the water-soluble active component is not particularly limited and, for example, may be 0.001% by mass or greater, 0.005% by mass or greater, 0.01% by mass or greater, 0.05% by mass or greater, 0.1% by mass or greater, 0.5% by mass or greater, 1.0% by mass or greater, 1.5% by mass or greater, 2.0% by mass or greater, 3.0% by mass or greater, 4.0% by mass or greater, or 5.0% by mass or greater, and may be 90% by mass or less, 80% by mass or less, 70% by mass or less, 60% by mass or less, 50% by mass or less, 40% by mass or less, 30% by mass or less, 20% by mass or less, 10% by mass or less, 8.0% by mass or less, 5.0% by mass or less, 3.0% by mass or less, 2.0% by mass or less, or 1.0% by mass or less.

### (Additional Components)

The composition of the present invention may further comprise, as additional components, one or more components generally used in external preparations for skin such as cosmetics and pharmaceuticals, in addition to the polymer of the present invention and the water-soluble active component described above.

Specifically, examples of the additional components include powder components, liquid oils and fats, solid oils and fats, waxes, hydrocarbons, higher fatty acids, higher alcohols, esters, silicones, anionic surfactants, cationic surfactants, amphoteric surfactants, nonionic surfactants, water-soluble polymers, thickeners, film-forming agents, UV absorbers, sequestering agents, lower alcohols, polyhydric alcohols, saccharides, amino acids, organic amines, polymer emulsions, pH adjusters, skin nutrients, vitamins, antioxidants, antioxidant aids, perfumes, and water, but are not limited thereto.

### (Dosage Form and Product Form of Composition)

The dosage form of the composition of the present invention is not particularly limited and, for example, may be a solution system, a solubilized system, an emulsion system, a powder dispersion system, a water-oil two-layer system, a water-oil-powder three-layer system, a gel, a mist, a spray, a mousse, a roll-on, or a stick, or may be a preparation impregnated or applied to a sheet such as a nonwoven fabric.

The product form of the composition of the present invention is not particularly limited and, for example, may be a facial cosmetic such as a toner, an emulsion, a cream, or a pack; a makeup cosmetic such as a foundation, a lipstick, or an eyeshadow; a sunscreen cosmetic (sunscreen agent); a body cosmetic; a fragranced cosmetic; a skin cleanser such as a makeup remover or a body shampoo; a hair cosmetic such as a hair liquid, a hair tonic, a hair conditioner, a shampoo, a rinse, or a hair growth treatment; or an ointment.

### <Application of Polymer of Present Invention>

The present invention also provides use of the polymer of the present invention described above as a percutaneous penetration enhancer of a water-soluble active component.

Regarding the details of the water-soluble active component and the blending amount of the polymer of the present invention described above when using the polymer as a percutaneous penetration enhancer of a water-soluble active component, the section “Composition of present invention” described above can be appropriately referenced.

### EXAMPLES

The present invention will be further described in detail with reference to the Examples below. However, the present invention is not limited thereto.

### Synthesis Examples 1 and 2

In each of the following Synthesis Examples 1 and 2, a polymer having a structure represented by formula (3) below was synthesized:

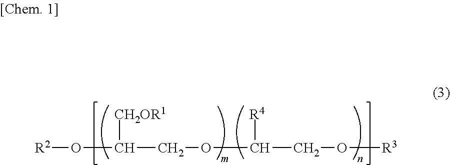

Note that in the formula (3), within the [ ] is a random copolymer.

### Synthesis Example 1

Synthesis Example 1: Polymer 1, where R¹=H; R²=H; R³=CH₃; R⁴=CH₃; m=2.0; and n=6.0

Synthesis was carried out by the method below to obtain Polymer 1.

128 g of methanol and, as a catalyst, 6.4 g of potassium hydroxide were placed inside an autoclave, air inside the autoclave was replaced with dry nitrogen, and the catalyst was completely dissolved at 80° C. while stirring. Next, a mixture of 592 g of glycidol and 1392 g of propylene oxide was added dropwise at 95° C. over 20 h with a dropping apparatus, followed by stirring for 5 h. The reaction composition was taken out from the autoclave, neutralized with hydrochloric acid to a pH of 6 to 7, and treated under reduced pressure of −0.095 MPa (50 mmHg) at 100° C. for 1 h to remove contained moisture. After the treatment, filtration was further carried out to remove any salt generated, and 2000 g of Polymer 1 of Synthesis Example 1 was obtained.

Various physical property values for the obtained Polymer 1 were determined below.

### (Number Average Molecular Weight)

The number average molecular weight of the obtained Polymer 1 was calculated by gel permeation chromatography (GPC) measurement. Using a SHODEX™ GPC101 dedicated GPC system as the system, a SHODEX RI-71s as the differential refractometer, a SHODEX KF-G as the guard column, and three SHODEX KF804L installed in series as columns, tetrahydrofuran as the developing solvent was flowed at a flow rate of 1 ml/min at a column temperature of 40° C. 0.1 ml of a 0.1 wt % tetrahydrofuran solution of the resulting reaction product was injected, and a chromatogram represented by refractive index intensity and elution time was obtained using a BORWIN GPC calculation program. A number average molecular weight was determined from this chromatogram using polyethylene glycol as the standard, and was about 530.

### (Weight Average Molecular Weight)

The weight average molecular weight of the obtained Polymer 1 was determined by GPC calculation in the same manner as above, and was about 859.

### (Polydispersity)

From the results of number average molecular weight and weight average molecular weight determined above, the polydispersity Mw/Mn of Polymer 1 was 1.62.

### (IOB Value)

The IOB value of the obtained Polymer 1 was determined as follows, and was 1.1.

More specifically, in Polymer 1, the organic value was calculated based on 20 for carbon and −10 for an iso-branch. In addition, the inorganic value was calculated based on 100 for a hydroxyl group and 20 for an ether bond.
Methanol site: (1 carbon atom, 1 hydroxyl group)×1
   Organic value: 20
   Inorganic value: 100
Glycidol site: (3 carbon atoms, 1 iso-branch, 1 hydroxyl group, 1 ether bond)×2

$$Organic value : ( 60 - 10 ) \times 2 = 100$$

$$Inorganic value : ( 100 + 20 ) \times 2 = 240$$

Propylene oxide site: (3 carbon atoms, 1 iso-branch, 1 ether bond)×6

$$Organic value : ( 60 - 10 ) \times 6 = 300$$

$$Inorganic value : 20 \times 6 = 120$$

From the above, the total organic value is 420 and the total inorganic value is 460, and thus the IOB value is 1.09.

### (Cloud Point)

A 5 wt % aqueous solution of the obtained Polymer 1 was prepared, heated to 85° C., and then cooled. The temperature at which a transparent solution was formed was 74° C. Thus, it was found that the cloud point of Polymer 1 was 74° C.

### (Hydroxyl Value)

The hydroxyl value of the obtained Polymer 1 was measured in accordance with the measurement method in JIS K-1557-1, and was 294.

### (Solubility in Water)

Solubility in water of the obtained Polymer 1 was determined by the same method as that for “Compatibility evaluation” described below, and was 50% by mass or greater.

### (Solubility in Olive Oil)

Solubility in olive oil of the obtained Polymer 1 was determined as follows. Specifically, Polymer 1 was mixed with olive oil so as to have a concentration of 1.0 wt %, and appearance was confirmed. “Yes” was indicated if the appearance was uniform. In this case, the solubility in olive oil of Polymer 1 was 1.0% by mass.

Various physical property values of Polymer 1 measured below are shown in Table 1 below.

### Synthesis Example 2

Synthesis Example 2: Polymer 2, where R¹=H and CH₃; R²=H and CH₃; R³=CH₃; R⁴=CH₃; m=2.0; and n=6.0

Synthesis by the method below was carried out to obtain Polymer 2.

128 g of methanol and, as a catalyst, 6.4 g of potassium hydroxide were placed inside an autoclave, air inside the autoclave was replaced with dry nitrogen, and the catalyst was completely dissolved at 80° C. while stirring. Next, a mixture of 592 g of glycidol and 1392 g of propylene oxide was added dropwise at 95° C. over 20 h with a dropping apparatus, followed by stirring for 2 h. Next, 244 g of potassium hydroxide was added, and the inside of the system was replaced with dry nitrogen. After the inside of the system was replaced with dry nitrogen, 200 g of methyl chloride was injected at a temperature of 80 to 130° C. and reacted for 5 h. The reaction composition was then taken out from the autoclave, neutralized with hydrochloric acid to a pH of 6 to 7, and treated under reduced pressure of −0.095 MPa (50 mmHg) at 100° C. for 1 h to remove contained moisture. After the treatment, filtration was further carried out to remove any salt generated, and 1820 g of Polymer 2 was obtained. A sample was taken before reacting the methyl chloride, and the hydroxyl value of the purified polymer was 125. Thus, it was found that the ratio of methyl group to hydrogen atom (CH₃/H) of R² and R³ was 0.57. Specifically, the hydroxyl group blocking rate of Polymer 2 was 57%.

In the same manner as in the case of Polymer 1 mentioned above, various physical property values for the obtained Polymer 2 were determined, and each of the results is shown in Table 1 below.

TABLE 1
(Table 1 Synthesis Example 1 and Synthesis Example 2)

| Synthesis Example No. | | |
|---|---|---|
| (Polymer No.) | 1 | 2 |
| | 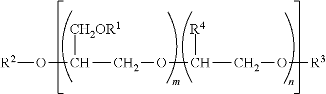 | |
| R¹ | H | CH₃ and H |
| R² | H | CH₃ and H |
| R³ | CH₃ | CH₃ |
| R⁴ | CH₃ | CH₃ |
| Ratio of CH₃ to H in R¹ and R² (CH₃/H) | — | 0.57 |
| m | 2.0 | 2.0 |
| n | 6.0 | 6.0 |
| m + n | 8.0 | 8.0 |
| Polymerization type | Random | Random |
| Number average molecular weight | 530 | 673 |
| Weight average molecular weight | 859 | 956 |
| Polydispersity (Mw/Mn) | 1.62 | 1.42 |
| IOB value | 1.09 | 0.77 |
| Hydroxyl group blocking rate (%) | — | 57 |
| Hydroxyl value (mgKOH/g) | 294 | 125 |
| Cloud point (5% aq.) | 74° C. | 45° C. |
| Solubility (water) | 50% by mass or greater | 30% by mass |
| Solubility (oil) *1 | Yes | Yes |

*1 Synthesized compound was mixed with olive oil so as to have a concentration of 1.0% by mass, and appearance was confirmed. “Yes” was indicated if appearance was uniform.

### <Compatibility Evaluation>

Polymers 1 and 2 synthesized above were each mixed with water and various oils at certain concentrations, stirred, and then left to stand for one day, followed by examining individual compatibility by visual observation. Results thereof are shown in Table 1-2 below. Note that in Table 1-2, “Yes” represents “compatible” and “No” represents “incompatible”.

TABLE 1-2

| | Concentration | | |
|---|---|---|---|
| | in each polymer | | |
| | (% by mass) | Polymer 1 | Polymer 2 |
| Water | 30 | Yes | Yes |
| | 50 | Yes | No |
| Squalane | 1 | No | No |
| Hydrogenated polyisobutene | 1 | No | No |
| Olive oil | 1 | Yes | Yes |
| Cetyl ethylhexanoate | 1 | No | No |
| Macadamia seed oil | 1 | Yes | Yes |
| Isopropyl palmitate | 1 | No | Yes |
| Isopropyl myristate | 1 | No | Yes |
| Ethyl hexyl palmitate | 1 | No | No |

### Example 1, Example 2, and Comparative Example 1

Based on the formulations in Table 2, each of the beauty compositions of the Examples and Comparative Examples was prepared. For each of the prepared compositions, a “cumulative permeation test” of each water-soluble active component into the skin was carried out. Note that the numerical values in Table 2 and Tables 3 and 4 described below and the numerical values, other than pH values, in Table 5 refer to blending amounts of components, expressed in % by mass.

TABLE 2

| | | | | Comparative |
|---|---|---|---|---|
| Configuration | | Example 1 | Example 2 | Example 1 |
| Percutaneous penetration | Polymer of present invention | Polymer 1 | Polymer 2 | — |
| enhancer | Blending amount | 5.0 | 5.0 | — |
| Water-soluble active | Niacinamide | 5.0 | 5.0 | 5.0 |
| component | (logP value: −0.4) | | | |
| Additional component | Ion-exchange water | balance | balance | balance |

| | | | |
|---|---|---|---|
| Total | 100 | 100 | 100 |

### <Cumulative Permeation Test>

A vertical Franz diffusion cell (hereinafter, referred to as “Franz cell”), which allows for the sampling over time regarding the permeability of each prepared composition and is also described in the Japanese Pharmacopoeia, was used to evaluate artificial skin membrane permeability of each water-soluble drug composition.

More specifically, a Franz cell having an effective permeation area of 1.77 cm² was used as the Franz cell. As an artificial skin membrane that is a permeable membrane arranged in the Franz cell, a Strat-M™ membrane (manufactured by Merck Millipore), which was cut out with a punch to match the outer diameter of the donor cell constituting the Franz cell, was used. The artificial skin membrane was sufficiently hydrated by immersing in physiological phosphate-buffered saline (PBS) before being set in the Franz cell. A water bath and a receiver cell jacket were connected to the Franz cell with hoses to assemble an integrated thermostatic system, and the receiver cell was then filled with PBS. Using a cell clamp, the artificial skin membrane was firmly fixed between the receiver cell and the donor cell to prevent air from entering. The surface temperature of the artificial skin membrane was maintained at about 32° C., which corresponds to the surface temperature of the skin, using a temperature regulator, and the PBS within the receiver cell was equilibrated for 1 h or more while being stirred with a stirrer bar. Application of each composition to the artificial skin membrane was carried out using a controlled open application method, assuming actual use. Each composition was applied uniformly to the artificial skin membrane for 30 s. After a predetermined time had elapsed since application (after 0, 1, 2, 4, 8, and 24 h had elapsed), an extract was collected from the receiver cell. Using an HPLC system provided with an LC-MS detector, the drug amount in the collected extract was quantified to calculate a cumulative permeation amount.

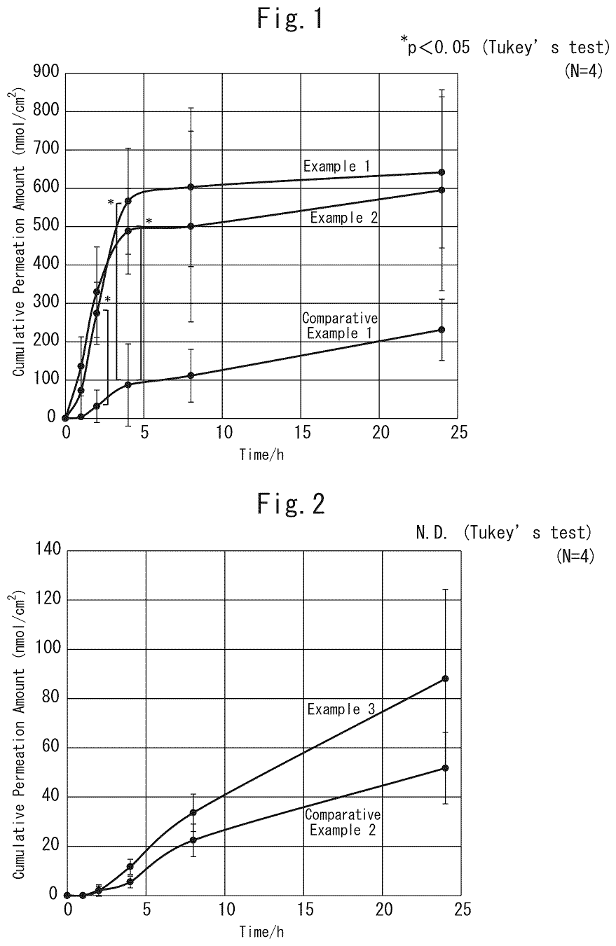

FIG. 1 shows the results of cumulative permeation amounts of the water-soluble active component (niacinamide) of Example 1, Example 2, and Comparative Example 1.

As is clear from the results in FIG. 1, it was found that niacinamide penetrated more rapidly in both Example 1 containing Polymer 1 and Example 2 containing Polymer 2 than in Comparative Example 1 not containing Polymer 1 or Polymer 2, and the cumulative permeation amount in both Example 1 and Example 2 increased significantly.

### Example 3 and Comparative Example 2

Based on the formulations in Table 3, each of the beauty compositions of Example 3 and Comparative Example 2 was prepared. For each of the prepared compositions, a “cumulative permeation test” of each water-soluble active component into the skin was carried out.

TABLE 3

| | | | Comparative |
|---|---|---|---|
| Configuration | | Example 3 | Example 2 |
| Percutaneous penetration | Polymer of present invention | Polymer 2 | — |
| enhancer | Blending amount | 5.0 | — |
| Water-soluble active | Potassium 4-methoxysalicylate | 1.0 | 1.0 |
| component | (ionic substance) | | |
| Additional component | Ion-exchange water | balance | balance |

| | | |
|---|---|---|
| Total | 100 | 100 |

FIG. 2 shows the results of cumulative permeation amounts of the water-soluble active component (potassium 4-methoxysalicylate) of Example 3 and Comparative Example 2.

As is clear from the results in FIG. 2, it was found that potassium 4-methoxysalicylate penetrated more rapidly in Example 3 containing Polymer 2 than in Comparative Example 2 not containing Polymer 2, and the cumulative permeation amount in Example 3 increased significantly.

### Example 4, Example 5, and Comparative Example 3

Based on the formulations in Table 4, each of the beauty compositions of Examples 4 and 5 and Comparative Example 3 was prepared. For each of the prepared compositions, a “cumulative permeation test” of each water-soluble active component into the skin was carried out.

TABLE 4

| | | | | Comparative |
|---|---|---|---|---|
| Configuration | | Example 4 | Example 5 | Example 3 |
| Percutaneous penetration | Polymer of present invention | Polymer 1 | Polymer 2 | — |
| enhancer | Blending amount | 5.0 | 5.0 | — |
| Water-soluble active | 1-piperinepropionic acid | 3.0 | 3.0 | 3.0 |
| component | (logP value: −1.5) | | | |
| Additional component | Ion-exchange water | balance | balance | balance |

| | | | |
|---|---|---|---|
| Total | 100 | 100 | 100 |

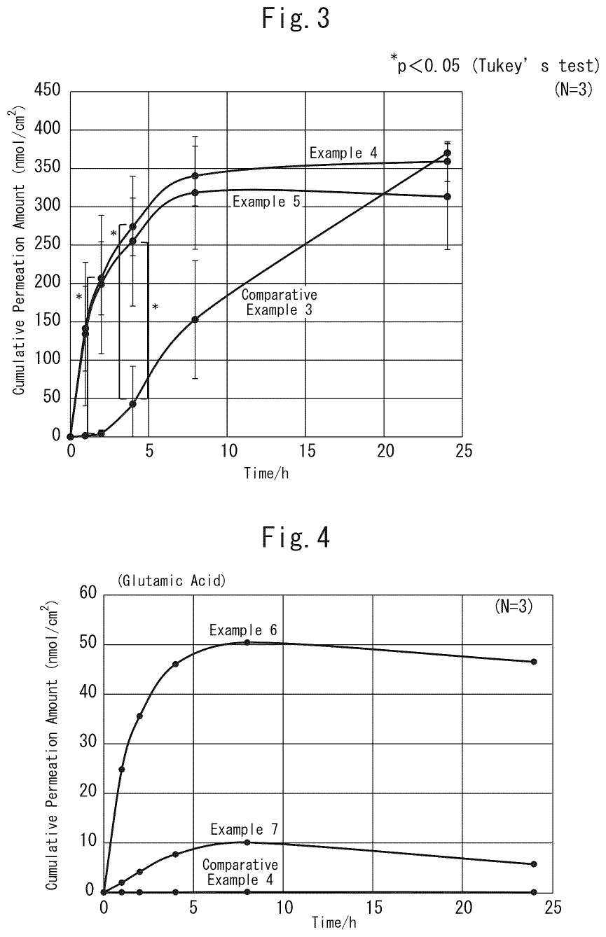

FIG. 3 shows the results of cumulative permeation amounts of the water-soluble active component (1-piperidinepropionic acid) of Examples 4 and 5 and Comparative Example 3.

As is clear from the results in FIG. 3, it was found that 1-piperidinepropionic acid penetrated more rapidly in both Example 4 containing Polymer 1 and Example 5 containing Polymer 2 than in Comparative Example 3 not containing Polymer 1 or Polymer 2, and the cumulative permeation amount in both Example 4 and Example 5 increased significantly.

### Examples 6 and 7 and Comparative Example 4

Based on the formulations in Table 5, each of the beauty compositions of Examples 6 and 7 and Comparative Example 4 was prepared. For each of the prepared compositions, a “cumulative permeation test” of each water-soluble active component (amino acid or derivative thereof) into the skin was carried out.

TABLE 5

| | | | | Comparative |
|---|---|---|---|---|
| Configuration | | Example 6 | Example 7 | Example 4 |
| Percutaneous penetration | Polymer 1 | 2.0 | — | — |
| enhancer | Polymer 2 | — | 2.0 | — |
| Water-soluble active | D-glutamic acid*¹ | 0.5 | 0.5 | 0.5 |
| component | (logP value: −3.7) | | | |
| | DL-alanine*² | 0.5 | 0.5 | 0.5 |
| | (logP value: −3.0) | | | |
| | | 0.5 | 0.5 | 0.5 |
| | DL-methionine*³ | | | |
| | (logP value: −1.9) | | | |
| Additional component | 1% KOH | 18.7 | 18.7 | 18.7 |
| | Ion-exchange water | balance | balance | balance |

| | | | |
|---|---|---|---|
| Total | 100 | 100 | 100 |
| pH (room temperature) | 6.37 | 6.36 | 6.29 |

*¹Molecular formula of “D-glutamic acid” is C₅H₉NO₄, and the molecular weight is 147.13.
*²Molecular formula of “DL-alanine” is C₃H₇NO₂, and the molecular weight is 89.09.
*³Molecular formula of “DL-methionine” is C₅H₁₁NO₂S, and the molecular weight is 149.21.

FIG. 4 is a chart showing the cumulative permeation amounts of D-glutamic acid contained in the water-soluble active component of Example 6, Example 7, and Comparative Example 4.

As is clear from the results in FIG. 4, it was found that D-glutamic acid penetrated more rapidly in both Example 6 containing Polymer 1 and Example 7 containing Polymer 2 than in Comparative Example 4 not containing Polymer 1 or Polymer 2, and the cumulative permeation amount in both Example 6 and Example 7 increased significantly. Particularly in the case of Example 6, it was found that the effect of enhancing penetration of D-glutamic acid was significant.

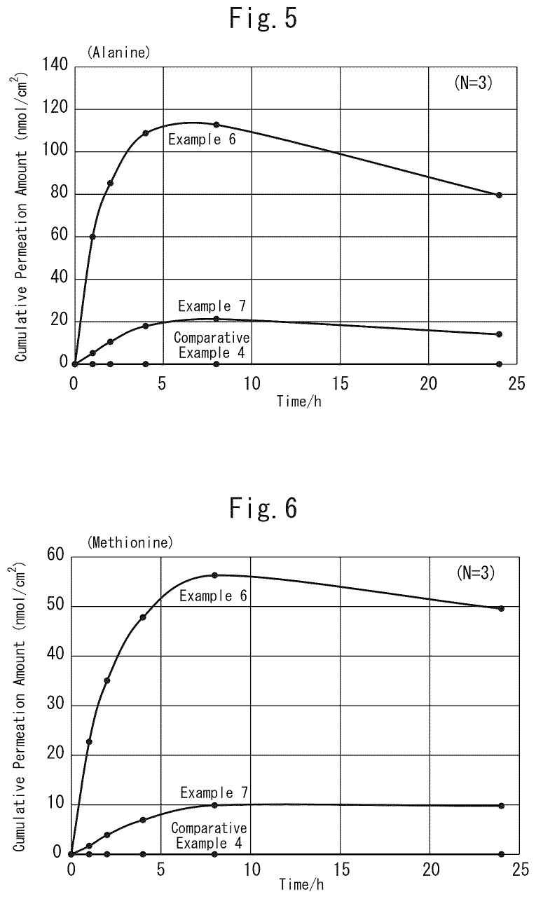

FIG. 5 shows the results of cumulative permeation amounts of DL-alanine contained in the water-soluble active component of Example 6, Example 7, and Comparative Example 4.

As is clear from the results in FIG. 5, it was found that DL-alanine penetrated more rapidly in both Example 6 containing Polymer 1 and Example 7 containing Polymer 2 than in Comparative Example 4 not containing Polymer 1 or Polymer 2, and the cumulative permeation amount in both Example 6 and Example 7 increased significantly. Particularly in the case of Example 6, it was found that the effect of enhancing penetration of DL-alanine was significant.

FIG. 6 shows the results of cumulative permeation amounts of DL-methionine contained in the water-soluble active component of Example 6, Example 7, and Comparative Example 4.

As is clear from the results in FIG. 6, it was found that DL-methionine penetrated more rapidly in both Example 6 containing Polymer 1 and Example 7 containing Polymer 2 than in Comparative Example 4 not containing Polymer 1 or Polymer 2, and the cumulative permeation amount in both Example 6 and Example 7 increased significantly. Particularly in the case of Example 6, it was found that the effect of enhancing penetration of DL-methionine was significant.

Glutamic acid, alanine, and methionine used as the water-soluble active components all have low log P values and generally have properties that hinder percutaneous penetration (refer to, for example, results of Comparative Example 4 in FIGS. 4 to 6). Therefore, it is clear that the effect of enhancing penetration of these water-soluble active components in Example 7 is somewhat inferior to that in Example 6, but shows an advantageous effect compared to that in Comparative Example 4.

1. A composition that is a cosmetic composition comprising a polymer having a structure represented by formula (1) below and a water-soluble active component, 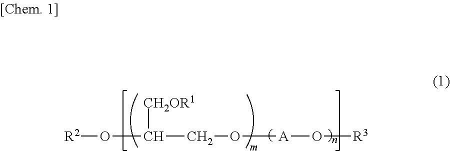
where in the formula (1), R¹, R², and R³ are each independently a hydrogen atom or an alkyl group having 1 to 4 carbon atoms; A is an alkylene group having 2 to 4 carbon atoms; and m and n are each independently 1.0 to 50,
wherein a number average molecular weight of the polymer is 10,000 or less, and
wherein an IOB value of the polymer is 0.4 to 1.8.

2. The composition according to claim 1, wherein
in the formula (1), A is represented by formula (2) below: 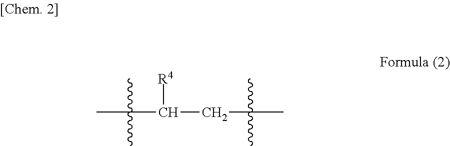
where in the formula (2), R⁴ is an alkyl group having 1 or 2 carbon atoms.

3. The composition according to claim 1, wherein the polymer is a random copolymer.

4. The composition according to claim 1, wherein in the formula (1), m is 2 or greater and at least a portion of R¹ includes a methyl group.

5. The composition according to claim 1, wherein in the formula (1), at least one of R² and R³ is a methyl group.

6. The composition according to claim 1, wherein the water-soluble active component comprises an ionic substance, or a nonionic substance having a log P value of less than 2.0.

7. The composition according to claim 1, wherein the water-soluble active component comprises at least one selected from a group consisting of alkoxysalicylic acid or salts thereof, 1-piperidinepropionic acid or salts thereof, B vitamins or derivatives thereof, and amino acids or derivatives thereof.

8. The composition according to claim 1, wherein the polymer is a percutaneous penetration enhancer of the water-soluble active component.

9. A method of enhancing percutaneous penetration of a water-soluble active component comprising contacting the water-soluble active component with a polymer having a structure represented by formula (1) below: 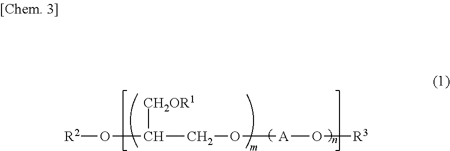
where in the formula (1), R¹, R², and R³ are each independently a hydrogen atom or an alkyl group having 1 to 4 carbon atoms; A is an alkylene group having 2 to 4 carbon atoms; and m and n are each independently 1.0 to 50,
wherein a number average molecular weight of the polymer is 10,000 or less, and
wherein an IOB value of the polymer is 0.4 to 1.8.

10. The method according to claim 9, wherein
in the formula (1), A is represented by formula (2) below: 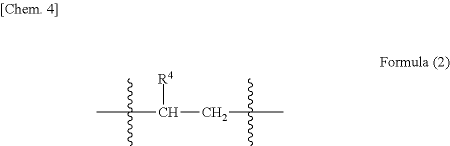
where in the formula (2), R⁴ is an alkyl group having 1 or 2 carbon atoms.

11. The method according to claim 9, wherein the polymer is a random copolymer.

12. The method according to claim 9, wherein in the formula (1), m is 2 or greater and at least a portion of R¹ includes a methyl group.

13. The method according to claim 9, wherein in the formula (1), at least one of R² and R³ is a methyl group.

14. The method according to claim 9, wherein the water-soluble active component comprises an ionic substance, or a nonionic substance having a log P value of less than 2.0.

15. The method according to claim 9, wherein the water-soluble active component comprises at least one selected from a group consisting of alkoxysalicylic acid or salts thereof, 1-piperidinepropionic acid or salts thereof, B vitamins or derivatives thereof, and amino acids or derivatives thereof.
